(19) United States
(12) Patent Application Publication
(10) Pub. No.: US 2023/0045204 A1
(43) Pub. Date: Feb. 9, 2023

(54) SUPPORTING DEVICE FOR SUPPORTING A BODY PART OF A USER

(71) Applicant: CO12 GmbH, Lichtenfels (DE)

(72) Inventors: Jens Henzler, Lichtenfels (DE); Ingo Güttler, Lichtenfels (DE); Oliver Hofmann, Lichtenfels (DE)

(21) Appl. No.: 17/784,286

(22) PCT Filed: Dec. 16, 2020
(86) PCT No.: PCT/EP2020/086489

§ 371 Date: Jun. 10, 2022

(30) Foreign Application Priority Data
Dec. 19, 2019 (DE) 10 2019 135 329.7

(51) Int. Cl. A61H 1/02

(52) U.S. Cl. CPC A61H 1/0288; A61H 2201/1638; A61H 2201/1253; A61H 2230/04; A61H 2230/207; A61H 2201/5023; A61H 2201/0242; A61H 2201/169; A61H 2201/5097; G06F 3/016

(57) ABSTRACT

Support device for supporting at least one body part of a user at least in portions, optionally completely, comprising a support body, which comprises at least one support region for supporting at least one body part of a user at least in portions, optionally completely.

### CROSS REFERENCE TO RELATED APPLICATIONS

The present specification is a National Phase Entry of International Application No. PCT/EP2020/086489 filed Dec. 16, 2020 and entitled “Supporting Device For Supporting a Body Part of a User” which, itself, claims priority to DE 10 2019 135 329.7 filed Dec. 19, 2019, each of which are incorporated by reference herein in their entireties.

### FIELD

The present specification relates to a support device for supporting at least one body part of a user at least in portions, optionally completely, wherein the support device comprises a support body, which comprises at least one support region for supporting at least one body part of a user at least in portions, optionally completely.

### BACKGROUND

Corresponding support devices are known per se from the prior art in different configurations and typically comprise a support body, which comprises at least one support region for supporting at least one body part, e.g. a limb, of a user at least in portions, optionally completely.

The main function of corresponding support devices consists in mounting a body part supported on a corresponding support region of a support body of the support device. The support region is intended to be designed in such a way that a body part may be supported or mounted on the support region in a position that is as relaxed as possible, specifically to that body part; this may be expedient for relieving tension or influencing the internal tension (tone) of the muscles, optionally as part of therapy.

In this connection, there is a need to improve or develop known support devices, since the support bodies associated therewith are typically individually moulded bodies, such that the effort in terms of manufacturing to produce the supporting devices is relatively high owing to the large number of anatomically different body parts, even when considering just one particular type of body part, such as a hand.

### SUMMARY

On this basis, the object of the present specification is to provide a support device for supporting a body part of a user that is improved compared with the above.

The object is achieved by a support device according to claim 1. The claims that are dependent thereon relate to possible embodiments of the support device.

A first aspect of the present specification relates to a support device for supporting at least one body part of a user at least in portions, optionally completely. The support device therefore in general serves to support at least one body part of a user at least in portions, optionally completely. Wherever the term “user” is used below, this of course includes all genders.

The term “support” can be understood to mean mounting at least one body part or at least one portion of a body part. The support device can therefore be referred to as or considered to be a mounting device. The support body associated with the support device can accordingly be referred to as or considered to be a mounting body. The support region to be assigned to the support body can accordingly be referred to as or considered to be a mounting region.

The term “body part” can be understood both as a body part and as a portion of a body part. An example of a body part is the torso or a limb. An example of a portion of a body part is thus a torso portion or a limb portion.

The term “limb” can be understood both as a complete limb (extremity) and as a limb portion of a superordinate limb. An example of a limb is therefore an arm or a leg. An example of a limb portion is thus a hand or a foot. However, a hand or a foot may also be an example of a limb. An example of a limb portion may thus also be a finger or a toe.

It is clear from the above explanations that the support device can be configured in particular for supporting a limb of an upper extremity of a user, in particular a hand, a portion of the hand, an arm or a portion of an arm, or for supporting a limb of a lower extremity of a user, in particular a foot, a portion of a foot, a leg or a portion of a leg.

The support device typically serves to support or mount body parts, i.e. in particular limbs, or portions of body parts, i.e. in particular portions of limbs, of humans or the human body; however, the supporting or mounting of body parts or portions of body parts of an animal or animal body are also conceivable.

As mentioned, the support device comprises a support body. The support body, which, as also mentioned, can also be referred to as or considered to be a mounting body, comprises at least one support region, which, as mentioned, can also be referred to as or considered to be a mounting region, for supporting at least one body part, i.e. a limb, for example, of a user at least in portions, optionally completely. The support region is therefore the region of the support device on which a user (directly) supports a body part when the support device is used as intended. The support region therefore constitutes the actual region of the support device on which a user supports or mounts a body part when the support device is used as intended. The support region therefore typically forms at least one part of the (exposed) surface of the support device.

The support device therefore comprises an e.g. block-like or block-shaped support body that is designed to be configured in a predetermined way, which comprises at least one surface which has, at least in portions, optionally completely, three-dimensional contours or structures that are designed to be configured in a predefined way in respect of a particular body part of a particular user. These three-dimensional contours or structures are typically formed by the support region in this case. The support region can be formed in one piece with the support body. It is also conceivable that the support region be formed as a separate component, which is connected to or can be connected with the support body so as to form the supporting device.

The dimensions of the support device, respectively the support body, respectively the support region are typically adapted to the dimensions of the respective body part to be supported. When the support device is used as intended, the support region is therefore covered at least in portions, optionally completely by the body part being supported in each case.

The support device is characterised in that at least the support region has, at least in portions, optionally completely, a geometric-structural design or shape designed to be configured in a predefined way on the basis of data describing selected support device dimension information and body part dimension information describing the dimensions of a body part that is to be supported on the support region at least in portions, optionally completely, of a plurality of predefined support device dimension information, wherein each piece of support device dimension information of at least one dimension of a body part describes assigned predefined dimensions of a support device produced on the basis of the respective piece of support device dimension information. The support region therefore has a geometric-structural design or shape configured in a predefined way which is formed or generated on the basis of data described by a piece of support device dimension information.

The piece of support device dimension information used as a basis for the data forming or generating the support region is selected from a plurality of predefined support device dimension information, wherein each piece of support device dimension information describes predefined dimensions of a support device which can be produced on the basis of the respective piece of support device dimension information, wherein each piece of support device dimension information is assigned to at least one dimension, e.g. length, width, height, etc., or at least one dimension ratio, e.g. a length-width-ratio, a length-height-ratio, a width-height-ratio, a length-width-height-ratio, etc. of a body part. The selection of the piece of support device dimension information of the plurality of pieces of support device dimension information is carried out on the basis of a piece of body part dimension information describing the dimensions of a body part that is to be supported on the support device at least in portions, optionally completely.

The geometric-structural design or shape of the support region that is configured in a predefined way creates significant advantages in terms of manufacturing, provided the support region and typically also the entire support device are not an individually configured component portion or not an individually configured component. This is because the support region is configured in a predefined way, i.e. it has a specific spatial-physical design that is configured in a predefined way, and therefore a specific spatial form that is configured in a predefined way, which is not individually configured with regard to a body part that is to be supported on the support device, even though it typically comprises support portions formed in an abstracted or standardised shape that is therefore also configured in a predefined way with regard to certain characteristics of a respective body part that is to be supported, e.g. the number and arrangement of fingers of a (human) hand, the number and arrangement of toes of a (human) foot, etc. The same applies to the entire support device; especially when the support region is formed in one piece with the support body.

The support region or the support device thus does not have an individually configured geometric-structural design or customisation other than potential adjustments or scaling of the dimensions. The support region or the support device is therefore not an individual part that is individually configured for a particular body part of a particular user. Rather, the support region or the support device can be understood as a mass component that is easily produced in terms of manufacturing, with regard to specific dimensions of a body part, but otherwise entirely independent from individual characteristics of the respective body part. Due to the geometric-structural design or shape that is configured in a predefined way, the support region or the support device typically has at least in portions, optionally completely, a standardised geometric-structural design or shape, which is or is being adjusted only by scaling with regard to the dimensions of a body part to be specifically supported, if at all.

The geometric-structural design of the support region that is configured in a predefined way, with this in particular being understood to be three-dimensional contours or structures of the surface of the support region that are configured in a predefined way, typically constitutes an abstracted or standardised (negative) reproduction, of a body part that is to be supported on the support region. This abstraction or standardisation, despite of which it is not a fit of the support device that is individually configured in a user-specific or body part-specific manner, typically results in an accurate enough fit of the support region allowing a respective body part to be supported on the support region in a relaxing or relaxed position specific to this body part.

Supporting a body part in a relaxing or relaxed position specific to this body part can both have a relaxing effect on the respective body part as well as on further body parts adjacent to this body part, such that a user's whole body can be brought into a relaxing or relaxed state when using the support device. In addition to the mere relaxing effect or as a consequence thereof, where necessary, therapeutic effects can also be brought about, since supporting the body part in a relaxing or relaxed position specific to this body part can e.g. have a therapeutic effect on the blood flow and/or oxygen supply to the respective body part. Positive effects can be achieved in particular in rheumatic body parts or limbs and/or in e.g. spastic body parts or limbs due to a stroke. The increase in the internal tension (tone) of the muscles of the respective body part, associated with spasticity, can be reduced (significantly in some cases) by (regular) use of the support device.

The support device can be used in particular without any help from a third party; therefore, the user can independently carry out therapeutic measures, e.g. for regulating tone, pain therapy, etc., and prepare themselves for corresponding therapeutic measures in the sense of self-help by using the support device as intended.

Using the example of a spastic hand, the support device, which, in this respect, can be referred to as or considered to be a support for the heel of the hand for providing therapy to persons with a cramping or cramped hand, makes it possible for the hand to lie on the support device in a loosened state, whereby a state of relaxation is achieved.

As mentioned, the support region may be formed in one piece with the support body. In this first variant, the support region typically forms an (exposed) surface of the support body.

As also mentioned, it is, however, also conceivable for the support region to be designed as a component that is separate from the support body and, when required, is or can be connected, optionally in a detachable manner (without being damaged or destroyed), to the support body so as to form the support device. This second variant causes no disadvantages in terms of manufacturing since the support body as well as the support region are produced with a standardised design.

In the second variant, the support body and/or the support region may comprise suitable, optionally corresponding, connection elements, which allow for a stable, but nevertheless typically detachable (without being damaged or destroyed) connection of the support body to the support region, and vice versa. The support body may e.g. comprise at least one first connection element and the support region may comprise at least one second connection element, the at least one first and the at least one second connection element being configured to interact with one another so as to form the support device. The at least one first connection element and the at least one second connection element may e.g. be configured to interact with one another in a form-fitting and/or force-locked manner so as to form the support device. Alternatively or additionally it is also conceivable for the at least one first connection element and the at least one second connection element to be configured to interact with one another magnetically so as to form the support device.

The support region may comprise one or more support portions, which are in particular designed as receiving portions and are configured to receive a particular portion of a body part, in particular a limb portion, for example a finger or a toe, of a body part that is supported or is to be supported on the support region, i.e. in particular a limb. A support device intended to support a particular body part may therefore comprise a number of support portions that are configured in a predefined way specifically to this body part and as such support portions that are configured in a standardised way specifically to this body part. Respective support portions may e.g. be designed as receiving portions configured in a predefined or standardised way. A respective receiving portion configured in a predefined or standardised way may be designed for receiving one or more body parts. Typically, a respective receiving portion configured in a predefined or standardised way is designed for receiving (exactly) one particular body part.

The support body or the support region may comprise a specific number and/or arrangement of corresponding support portions configured in a predefined or standardised way which corresponds to the number and/or arrangement of the limb portions, i.e. the fingers or toes, for example, of a respective body part to be supported on the support region. Using the example of a hand to be supported on the support region, the support body or support region can therefore comprise e.g. several support portions, with each support-region portion supporting a particular finger of the hand to be supported. The same applies to other body parts.

The support body or the support region can in particular comprise at least one of a specific number and a specific arrangement of support portions, that are designed to be configured in a predefined way, which corresponds to the number and arrangement of limb portions, especially of fingers and toes, of a respective body part of a user which is to be supported on the support device.

If supporting a respective finger, corresponding support portions can be referred to as or considered to be finger support portions, and toe support portions if supporting a respective toe.

For supporting a hand, the support body or the support region may therefore comprise specifically at least two, in particular at least three, in particular at least four, optionally five finger support portions, designed to be configured in a particular way, in a parallel arrangement, in particular in a substantially parallel arrangement, corresponding to the number and arrangement of the fingers of a respective hand of a user which is to be supported on the support device. In such a way a particularly relaxing effect on a respective hand supported or to be supported on the support device is achievable. Respective finger support portions are also designed in a standardised way or configured in a predefined way. Respective finger support portions can be specifically designed e.g. as receiving portions or recess portions designed to be configured in a predefined or standardised way.

Respective finger support portions can be arranged and/or aligned extending in a first spatial direction, in particular a first distal spatial direction relative to a or the hand which is or is to be supported on the support device. In such a way, a particularly relaxing effect on a respective hand supported or to be supported on a support device is achievable. The same applies to other body parts.

The support device can further comprise or include at least one further support portion, in particular arranged or aligned transversely to the finger support sections, for receiving an end portion of a metacarpal region, i.e. in particular of a proximal end portion, of a hand of a user that is to be supported on the support device. In such a way, a particularly relaxing effect on a respective hand supported or to be supported on a support device is achievable. A corresponding further support portion if also designed to be configured in a predefined or standardised way. A corresponding further support portion can specifically be designed e.g. as a receiving portion or recess portion designed to be configured in a predefined or standardised way.

Respective finger support portions can transition into the further support portion, in particular directly. Respective finger support portions can therefore communicate with the further support portion, in particular directly. In such a way a particularly relaxing effect on a respective hand supported or to be supported on a support device is also achievable.

The support body or the support region can comprise or include a base portion which comprises one or several curvedly formed curved portions, in particular relative to a non-curved base plane, which are in particular modelled on a hand. Respective curved portions are also designed to be configured in a predefined or standardized way. Respective curved portions can specifically be designed as e.g. in particular pad-like or pad-shaped protrusions designed to be configured in a predefined or standardized way. In such a way, a particularly relaxing effect on a respective hand supported or to be supported on a support device is also achievable.

The base portion can in particular comprise at least one in particular pad-like or pad-shaped curved portion which is configured in a state of the support device, which is used as intended, for supporting a metacarpal region of a hand of a user that is or is to be supported on the support device. In such a way a particularly relaxing effect on a respective hand supported or to be supported on a support device is also achievable.

Respective finger support portions and/or at least one further support portion can be designed in e.g. at least one in particular ring segment-like or ring segment-shaped curvedly formed curve portion. In such a way a particularly relaxing effect on a respective hand supported or to be supported on a support device is also achievable.

Regardless of the number and/or arrangement of respective support portions, the support body or the support region can have a base form, configured in a predefined way, which is modelled on a body part that is to be supported on the support device. This can influence the usage behaviour in a positive way, as the base form of the support body or the support region already “invites”, in an intuitively detectable way, to support the respective body part; the base form of the support body or the support region can therefore positively influence the “user compliance”.

The support body can be arranged or designed on a base plate or carrier plate. The design or arrangement of the support body on a base plate or carrier plate ensures a stable support or mounting of the support device on a surface. Furthermore, a corresponding base plate or carrier plate can be equipped at least in portions, optionally completely, with at least one adhesion-increasing structuring such as a special surface structuring, e.g. a roughening, a rubber coating, etc., in order to ensure a stable support or mounting of the support device on a surface.

It is also conceivable that a corresponding base plate or carrier plate forms a member of a carrier device for carrying the support device. Along with a support body arranged or formed on it, a corresponding base plate or carrier plate can therefore be optionally (without damaging or destroying) detachable from, connected with, or connectable with e.g. a frame-like or frame-shaped carrier element by e.g. attachment or insertion thereof, if necessary, by forming a corresponding carrier device. The base plate or carrier plate or the carrier element can comprise suitable, optionally corresponding connection elements which ensure a stable though typically (without damaging or destroying) detachable connection of the base plate or carrier plate with the carrier element and vice versa. The base plate or carrier plate can comprise e.g. at least one first connection element and the carrier element at least one second connection element, the at least one first and the at least one second connection element being configured to interact with one another so as to form the carrier device. The at least one first connection element and the at least one second connection element can e.g. be configured to interact with one another in a form-fitting and/or force-locked manner so as to form the carrier device. It is alternatively or additionally conceivable that the at least one first connection element and the at least one second are configured to interact with one another magnetically so as to form the carrier device.

The support device may comprise a detection device, in particular integrated in the support body and/or the support region, which is configured to detect at least one chemical and/or physical variable of the support body and/or the support region and/or a body part supported on the support region. A corresponding detection device may be configured to e.g. detect chemical parameters, such as the composition of the surface of a supported body part or a substance on the surface of the supported body part, and/or thermal parameters, such as the temperature of the support body and/or the supported body part, and/or mechanical parameters, such as forces exerted on the support body and/or the support region, in particular by means of a supported body part, i.e. in particular weight forces, pressure, stress, etc., and/or hydrometric parameters, such as moisture of a supported body part, etc. A corresponding detection device may comprise one or more detection elements that are e.g. designed as or comprise sensors, which are arranged or formed on or in the support body and/or on or in the support region. The specific design of respective detection elements is determined by the respective chemical and/or physical variables to be detected. A corresponding detection device typically has suitable hardware-implemented and/or software-implemented evaluation and/or processing measures, i.e. suitable evaluation and/or processing algorithms, for example, which allow for data-related evaluation or processing of respective detection signals supplied by the detection elements.

The support device may further comprise a transmission device, which is or can be assigned to the detection device, which in particular is integrated in the support body and/or support region, and is configured to transmit a piece of detection information describing a chemical and/or physical variable detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

The support device may further comprise a detection device, in particular integrated in the support body and/or the support region, which is configured to detect a body part supported on the support region. A corresponding detection device may e.g. be configured to detect when a body part supported or to be supported on the support region approaches or contacts the support region or the support body. A corresponding detection device may comprise one or more detection elements that are e.g. designed as or comprise sensors, in particular proximity sensors or contact sensors, which are arranged or formed on or in the support body and/or on or in the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements. A corresponding detection device may, however, also be configured to be identical to the above-mentioned detection device for detecting a chemical and/or physical variable, since conclusions can optionally be drawn on a body part supported on the support region on the basis of respective detected chemical and/or physical variables.

The support device may further comprise a transmission device, which is or can be assigned to the detection device, which in particular is integrated in the support body and/or support region, and is configured to transmit a piece of detection information describing a body part supported on the support region detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

The support device may further comprise a detection device, in particular integrated in the support body and/or the support region, which is configured to detect at least one physiological variable, in particular the blood pressure, oxygen saturation or pulse, of a user supporting a body part on the support region. A corresponding detection device may comprise one or more detection elements that are e.g. designed as or comprise sensors, in particular blood-pressure, oxygen-saturation or pulse sensors, which are arranged or formed on or in the support body and/or on or in the support region. Corresponding physiological variables may e.g. be detected via the contact region between the support region and the respective body part supported on the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

The support device may further comprise a transmission device, which is or can be assigned to the detection device, in particular is integrated in the support body and/or support region, and is configured to transmit a piece of detection information describing a physiological variable detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

The support device may further comprise a detection device which is configured to detect a movement of the support body in at least one degree of freedom, in particular relative to a third object and/or relative to a surface, and/or to detect a movement of a body part supported on the support region in at least one degree of freedom, in particular relative to the support body. A corresponding detection device may comprise one or more detection elements that are e.g. designed as or comprise sensors, in particular motion sensors, which are arranged or formed on or in the support body and/or on or in the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

The support device may further comprise a transmission device, which is or can be assigned to the detection device, in particular is integrated in the support body and/or support region, and is configured to transmit a movement, detected by means of the detection apparatus, of the support body in at least one degree of freedom, in particular relative to a surface, and/or to transmit a movement, detected by means of the detection apparatus, of a limb supported on the support body in at least one degree of freedom, in particular relative to the support body, to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

The support device may further comprise a data storage device, in particular integrated in the support body and/or the support region, which is configured to store at least one piece of detection information detected by means of the detection device in terms of data. A corresponding data storage device is typically assigned a data-transmission interface, by means of which data can be read from the data storage device in a wired or wireless manner and/or by means of which data can be input into the data storage device.

The support device may further comprise a signal-output device, in particular integrated in the support body and/or the support region, which is configured to generate at least one in particular acoustic and/or optical and/or haptic output signal, in particular on the basis of at least one piece of detection information generated by the or a detection device. Certain output signals, i.e. generally signals, can therefore be output via a corresponding signal-output device by means of the support device. For example, an output signal indicating that a body part is properly supported on the support region can be output. A corresponding output signal may be generated or have been generated for example on the basis of detection information supplied by at least one detection device, i.e. a piece of detection information describing that a body part has been detected as contacting the support region, for example. A corresponding signal-output device typically comprises at least one signal-output element, which, depending on the specific signal to be output, may e.g. be designed as or comprise at least one acoustic signal-output element, i.e. a speaker, for example, as an optical signal-output element, i.e. a display element, light-emitting diode element, etc., for example, or as a haptic signal-output element, i.e. a vibration element, for example.

An output signal which can be output by means of a signal-output device, by way of example, may contain at least one piece of information relating to a user-side action and/or an evaluation of a user-side action, in particular on a user, of the support device. In this way, it is possible to instruct or assist a user in properly using the support device by means of corresponding output signals containing corresponding information. For example, it can be output to a user by corresponding output signals that the support device is ready to support a body part, and optionally also which body part, that a body part is properly supported or is not (yet) properly supported, or when/that a particular support duration, e.g. expedient for a therapeutic effect, has been reached or has not (yet) been reached, etc. Equally, output signals that encourage or motivate a user are conceivable, i.e. output signals that encourage or motivate a user, e.g. acoustically, optically or haptically, to use the support device, to assume and/or maintain a particular support position, etc. Similarly, output signals that assess, i.e. in particular praise or criticise, a user are conceivable, which assess a user or a particular user action, e.g. acoustically, optically or haptically, before and/or during and/or after use of the support device.

The support body may be integrated in an input apparatus of a device, i.e. a terminal, for example, such as a computer, a multimedia apparatus, etc., that can be controlled by an input apparatus, or is designed as an input apparatus of this kind. In this way, increased functional integration can be achieved, which also gives the user the option of carrying out particular actions, i.e. in particular inputs, controls, etc., using the body part while the body part is being supported. A specific example of a corresponding input apparatus is an input element, e.g. designed as an (electronic) computer mouse, for making inputs into a computer, an input element, e.g. designed as a remote control, for making inputs into a multimedia apparatus, such as an audio system or a television, etc. Other examples are conceivable.

The support device may comprise a temperature-control device, in particular integrated in the support body and/or the support region, for controlling the temperature of the support body and/or the support region at least in portions, optionally completely. The static or dynamic temperature control (understood in principle as both heating and cooling) of the support body and/or the support region, made possible by means of a corresponding temperature-control device, can assist the desired relaxation of a body part that is being supported or can assist a therapeutic effect that can be achieved by a body part being supported.

A corresponding temperature-control device may comprise a temperature-control duct structure comprising a temperature-control duct through which a temperature-control medium, i.e. a gas or liquid, for example, can flow. A corresponding temperature-control duct structure may extend through the support body or the support region, in particular in a planar manner, at least in portions, optionally completely, in one or several spatial directions, i.e. in particular in one or several horizontal and/or vertical spatial directions. In particular, a corresponding temperature-control duct structure may extend, in particular with close contours, in the region of or below the (exposed) surface of the support body or the support region forming an actual support surface of the support body or the support region.

Alternatively or additionally, a corresponding temperature-control device may comprise at least one electrically operated temperature-control element. A corresponding electrically operated temperature-control element may e.g. be designed as or comprise at least one heating wire or a one-dimensional or multi-dimensional heating-wire structure. A corresponding temperature-control element may extend through the support body or the support region, in particular in a planar manner, at least in portions, optionally completely. In particular, a corresponding temperature-control element may extend, in particular with close contours, in the region of or below the (exposed) surface of the support body or the support region forming an actual support surface of the support body or the support region.

Alternatively or additionally, a corresponding temperature-control device may comprise at least one temperature-control element, which performs an optionally reversible exothermic chemical reaction after activation. A corresponding temperature-control element may be arranged or formed in the region of or below the (exposed) surface of the support body or the support region forming an actual support surface of the support body or the support region. A corresponding temperature-control element may comprise an activation element, i.e. a tab, a button, etc., by means of which it is possible to activate the temperature-control element, i.e. to initiate the exothermic reaction.

The support device may further comprise or include a venting device, which is in particular integrated in the support body and/or support region, for ventilating at least in portions, optionally completely, a body part that is or is to be supported on the support device. The static or dynamic ventilation of the support body or the support region, which is made possible my means of a corresponding venting device, may support the desired relaxation of a supported body part, respectively a therapeutic effect achieved by supporting the body part. The support properties can furthermore be improved in particular in supporting a body part over a longer period.

A corresponding venting device may comprise a ventilation duct structure, which may comprise at least one ventilation duct, through which flows a ventilation medium, i.e. e.g. a gas. A corresponding ventilation duct structure may extend through the support body or the support region in particular in a planar manner, at least in portions, optionally completely. In particular, a corresponding ventilation duct structure may extend, in particular with close contours, in the region of or below the (exposed) surface of the support body or the support region forming an actual support surface of the support body or the support region.

A corresponding ventilation duct structure may be formed by one or more bionic geometries which are configured in a predefined way. Corresponding bionic geometries may support or improve the ventilation of a body part which is or is to be supported on the support device.

It is therefore generally possible to form bionic geometries, which are configured in a predefined way, in the support body in order to form a corresponding ventilation duct structure.

It shall be noted at this point that the support body may generally comprise at least in portions, optionally completely, a bionic geometry, which is configured in a predefined way.

The support device may further comprise or include a fixing device, which is or can be assigned to the support body and/or the support region, for temporarily fixing a body part supported on the support region, in particular in an intended support position of the body part on the support region. A corresponding fixing may facilitate and maintain a desired orientation of a body part supported on the support region. In this way, a relaxation effect or therapeutic effect that can be produced by the support device can be assisted. A corresponding fixing device can make it possible to fix a respective body part directly to the support body or the support region; this may e.g. be implemented by a fixing element that is or can be attached to the support body or the support region, such as a strap, a loop, etc., which surrounds the respective body part at least in portions, optionally completely, after being put on, such that the body part is fixed to the support body or the support region. A tube-like or tubular fixing device is also conceivable, i.e. a fixing tube made of a fabric, for example, which can be pulled over the support device and the body part that is supported or is to be supported thereon and, in this way, a desired orientation of a body part supported on the support region is facilitated and maintained. A fixing device is also conceivable which makes it possible to fix a superordinate body part, i.e. an arm, for example, in a particular orientation when the body part to be fixed is a hand.

The support device may further comprise or include an attaching device for temporarily attaching the support device to or on a third object, in particular to or on a third object to be worn on a body of a user of the support device. The support device may also be attached to third objects in such a way, i.e. in particular to mobile or portable third objects. Attachment of the support device to corresponding third objects can in particular be such that the support device may be in an orientation and/or position, in which a desired orientation of a body part supported on the support region is possible and can be maintained. A specific example for a third object is a third object, which is to be worn on a body of a user, such as a piece of clothing. The support device can therefore be attachable to or in a third object, such as a piece of clothing. It is, for example, conceivable, that the support device be attachable in a pocket of a piece of clothing, such as a piece of clothing which is to be worn on the upper body of a wearer, such as a jacket, a sweater, etc., or a piece of clothing to be worn on the lower body of a wearer, such as pants, a skirt, etc. The attaching device may comprise one or more attaching elements which are configured for attachment (without being damaged or destroyed) of the support device to a corresponding third object. Corresponding attaching elements may e.g. allow attaching the support device to a corresponding third object in a form-fitting and/or force-locked manner. A specific example for corresponding attachment elements are therefore hook and/or loop elements (Velcro fastener elements).

The support body may comprise at least one mounting portion for mounting the support body on a surface or a base. Depending on the design, the support body can be mounted in a stable manner on a surface or a base, such as a table surface, by means of a corresponding mounting portion.

A corresponding mounting portion may be provided with or comprise at least one material or corresponding material structure that provides non-slip support of the support body on a surface or a base. For example, a corresponding material or a corresponding material structure may therefore be expedient for allowing the support body to be supported in a stable manner on a surface or a base. A corresponding material or a corresponding material structure may e.g. be made of an elastomer material or an elastomer material structure or may comprise at least one of such. In principle, both natural and synthetic elastomer materials or material structures are possible. In terms of manufacturing, thermoplastic elastomers may advantageously be used, for example.

A corresponding mounting portion may be provided with or comprise at least one reversibly deformable material, in particular a reversibly deformable, flexible material, or a reversibly deformable material structure, in particular a reversibly deformable, flexible material structure. For example, a corresponding material or a corresponding material structure may therefore be expedient to allow any required modifications to the orientation of the support body on a surface or a base, in particular relative to a neutral or reference orientation, which, for example, can make it easier or make it possible for users with spastic body parts or limbs to use the support device; this is in particular applicable if the spasticity in question or another anatomical malalignment requires a particular orientation of the support body, in particular relative to a neutral or reference orientation, in order to support the body part on the support region, in particular without causing any pain. A corresponding material or a corresponding material structure may e.g. be made of an elastomer or cellular material or an elastomer or cellular material structure or may comprise at least one elastomer material. In principle, both natural and synthetic elastomer or cellular materials or material structures are possible. In terms of manufacturing, optionally foamed, thermoplastic elastomers may advantageously be used, for example.

A corresponding mounting portion may comprise at least one mounting-portion element which in particular lies in a non-planar manner on a planar surface or base and makes it possible to mount the support body that is mounted on the surface relative to the surface or base so as to be movable, in particular pivotally movable, in at least one degree of freedom. In this way, the support body can be moved relative to a surface or a base, which may be advantageous for producing the most relaxed possible position of a respective body part and superordinate body parts. For example, an orientation of the support body that can be modified compared with a reference orientation allows for (yet) more relaxing or relaxed support of a body part. A corresponding mounting-portion element may e.g. have a monogonal, polygonal, circular, round or pointed geometry. Likewise, movably mounting the support body can provide training options, e.g. for building muscle, since by moving the support body relative to the surface or the base, certain movement sequences associated with a training effect can be performed.

A corresponding mounting-portion element may also be designed as a separate part and, when required, is or can be connected, optionally in a detachable manner (without being damaged or destroyed), to the support body so as to form the support device. A corresponding, e.g. spherical, hemispherical, cylindrical or semi-cylindrical or pyramidal mounting-portion element may e.g. be connected to the support body by a hook-and-loop fastener system or an interchangeable fastener system.

The support device may comprise at least one support element, which is or can be supported on the support region, in particular detachably (without being damaged or destroyed), so as to cover said support region at least in portions, optionally completely. A corresponding support element may in particular be expedient from a hygiene viewpoint, since the support region or the support body is protected against direct contact with the respective body part and thus e.g. against deposits of skin, sweat, etc. A corresponding support element may be made of a resiliently flexible material or a resiliently flexible material structure, such that the support element does not impair the fit of the support region. A corresponding support element may be made of an optionally washable, natural and/or synthetic fabric material or a natural and/or synthetic fabric material structure. Merely by way of example, reference is made to leather or plastics materials.

The support body and/or the support region may be made of at least one foam material, i.e. in particular of an expanded particle foam material, such as an expanded plastic material, or comprise one of such. Corresponding foam materials reduce the weight of the support body or the support region. Corresponding foam materials may additionally cause pleasant haptic properties of the support body or support region. Further advantages of corresponding foam materials is the well manageable, cost-efficient machining of such.

Alternatively or additionally, the support body and/or the support region may be made of or comprise at least one magnetisable or magnetic material. Magnetisable or magnetic materials, such as corresponding ferromagnetic metals, may have a positive effect on the blood flow through the corresponding body part, and this can promote therapeutic effects. Magnetisable or magnetic materials may be provided as a solid material or in the form of particles.

Alternatively or additionally, the support body and/or the support region may be made of or comprise at least one antibacterial material. Antibacterial materials, such as antibacterial metals, such as silver, may have a positive effect on the hygienic properties of the support region and/or the support body. Antibacterial materials may be provided as a solid material or in the form of particles.

With regard to the production of the support device, i.e. the support body and/or the support region, in principle any manufacturing methods, in particular moulding and/or shaping manufacturing methods, are taken into consideration.

In particular, both additive (build-up) manufacturing methods as well as subtractive (material-removing) manufacturing methods, i.e. in particular machining manufacturing methods as well as shaping manufacturing methods, such as deep drawing, are taken into consideration.

Additive manufacturing methods may be expedient, since these are suitable for producing corresponding, geometric-structural designs configured in a predefined or standardised way. Therefore, additive manufacturing methods can be taken into consideration which allow for the additive build-up of three-dimensional objects by successively selectively hardening, in layers, an optionally powdered construction material that can be successively selectively hardened in layers (this may e.g. be a metal or a plastics material). Merely by way of example, in this connection reference is made to binder-jetting methods, selective laser-melting methods, selective laser-sintering methods or stereolithography methods.

It shall be noted at this point that the support device can be generally manufactured of any material. Plastics, metals, ceramics are referred to merely as examples. A forming of a composite material containing several different materials or material structures, i.e. differing in at least one chemical and/or physical parameter, is conceivable.

A second aspect of the present specification relates to a communication system which comprises at least one support device as described herein, which comprises a transmission device, which is configured to transmit a piece of information to at least one communication partner and/or to receive a piece of information transmitted by at least one communication partner, and at least one communication partner, which is configured to receive a piece of information transmitted by the at least one support device and/or to transmit a piece of information to the at least one support device.

All the embodiments relating to the support device, in particular all the embodiments relating to any transmission devices of the support device, apply by analogy to the communication system.

A third aspect of the present specification relates to a method for producing a receiving device for supporting at least one body part of a user at least in portions, optionally completely, wherein the method comprises the steps of:

providing body part dimension information describing the dimensions of a body part that is to be supported on the support region of the support device to be produced at least in portions, optionally completely, providing several pieces of support device dimension information in e.g. a data storage device, wherein each piece of support device dimension information describes at least one predefined dimension of a support device assigned to at least one dimension of a body part, which is producible or to be produced based on the respective piece of support device dimension information;
selecting a piece of support device dimension information based on the provided body part dimension information;

producing a support device based on the data including or describing the selected support device dimension information.

Corresponding data, on the basis of which the support device is produced according to the method, can therefore include production data of a production plant usable for or be used for producing the respective support device or be converted into such production data.

All the embodiments relating to the support device, in particular embodiments relating to manufacturing-related aspects of the support device, apply by analogy to the method.

The selection can be done according to the method by matching or comparing the provided piece of body part dimension information with respective body part dimension information assigned to respective support device dimension information, wherein a piece of support device dimension information can be chosen, whose assigned piece of body part dimension information is equal or similar, in particular within the scope of a tolerance range which can be or is predefined, to the provided piece of body part dimension information.

A corresponding matching can be made e.g. by matching at least one dimension described in the or a provided piece of body part dimension information, such as length, width, height, etc., of a body part or a body part portion or a dimension ratio such as a length-width-ratio, a body part or a body part portion, with corresponding dimensions described in at least the or a piece of support device dimension information, such as length, width, height, etc., or of a corresponding dimension ratio, such as a length-width-ratio. A corresponding matching may be made by a user, such as a wearer of the support device to be produced or a producer of the support device to be produced. Alternatively or additionally, a corresponding matching can be made, optionally in an automatable or automated manner, by a matching device implemented in terms of hardware and/or software and correspondingly configured in terms of software or hardware to perform a corresponding matching. A corresponding matching device can constitute a member, in terms hardware and/or software, of a superordinate mobile or stationary data processing device, which can therefore be configured to perform a corresponding matching.

A corresponding comparison can be made by comparing at least one dimension described in the or a provided piece of body part dimension information, such as length, width, height, etc., of a body part or a body part portion or a dimension ratio such as a length-width-ratio, a body part or a body part portion, with corresponding dimensions described in at least the or a piece of support device dimension information, such as length, width, height, etc., or of a corresponding dimension ratio, such as a length-width-ratio.

A piece of body part dimension information, which is to be provided or provided according to the method, may be done by detecting, in particular optically, a body part of a user that is to be supported on the support region. According to the method, an in particular optical detection of a body part of a user that is to be supported on the support device, as well as a generation of a piece of body part dimension information, which is to be provided, can therefore be made based on a piece of detection information generated by the detection. As implied, the detection can be made in particular optically, so that a detection can be made e.g. by means of a detection device in the form of a camera device or a video device. A corresponding camera device or video device constitute a member of a user- or producer-side, in particular mobile, end-device, such as a mobile computer (laptop), a smartphone, a tablet, smart glasses, etc.

Generally, a corresponding body part may e.g. be detected by means of a raw-data detection device, which is at least one in particular mechanical or optical detection device which is configured to detect one-dimensional or multi-dimensional body-part raw data describing the dimensions and/or morphology of at least one body part. A mechanical detection device may e.g. be designed as a dimension device, which makes it possible to detect body-part raw data describing the morphology of at least one body part on the basis of detected mechanical variables, i.e. in particular pressure. A mechanical detection device may in particular also be configured to create a body-part impression. An optical detection device may, as mentioned, e.g. be designed as a camera device, as a video device or as a scanning device, in particular as a body-part scanning device. In any case, it is also conceivable for a corresponding raw-data detection device to be designed as or comprise at least a detection device for in particular mechanically and/or optically detecting one-dimensional or multi-dimensional dimensions, i.e. the length, width, height, periphery or volume, of a body part.

### BRIEF DESCRIPTION OF THE DRAWINGS

The embodiments of the present specification are explained in greater detail herein with reference to the drawings, in which:

FIG. 1 is a schematic view of a support device for supporting a body part of a user according to an embodiment;

FIG. 2 is a schematic view of a support device for supporting a body part of a user according to an embodiment;

FIG. 3 is a schematic view of a support device for supporting a body part of a user according to an embodiment;

FIG. 4 is a schematic view of a support device for supporting a body part of a user according to an embodiment;

FIG. 5 is a schematic view of a support device for supporting a body part of a user according to an embodiment;

FIG. 6 is a schematic view of a support device for supporting a body part of a user according to an embodiment;

FIG. 7 is a schematic view of a support device for supporting a body part of a user according to an embodiment;

FIG. 8 is a schematic view of a support device for supporting a body part of a user according to an embodiment;

FIG. 9 is a schematic view of a support device for supporting a body part of a user according to an embodiment;

FIG. 10 is a schematic view of a support device for supporting a body part of a user according to an embodiment;

FIG. 11 is a schematic view of a support device for supporting a body part of a user according to an embodiment;

FIG. 12 is a schematic view of a support device for supporting a body part of a user according to an embodiment;

FIG. 13 is a schematic view of a support device for supporting a body part of a user according to an embodiment;

FIG. 14 is a schematic view of a support device for supporting a body part of a user according to an embodiment;

FIG. 15 is a schematic view of a support device for supporting a body part of a user according to an embodiment;

FIG. 16 is a schematic view of a support device for supporting a body part of a user according to an embodiment;

FIG. 17 is a schematic view of a communication system according to an embodiment; and

FIG. 18 is a schematic view of a method for producing a support device for receiving a body part of a user according to an embodiment.

### DETAILED DESCRIPTION

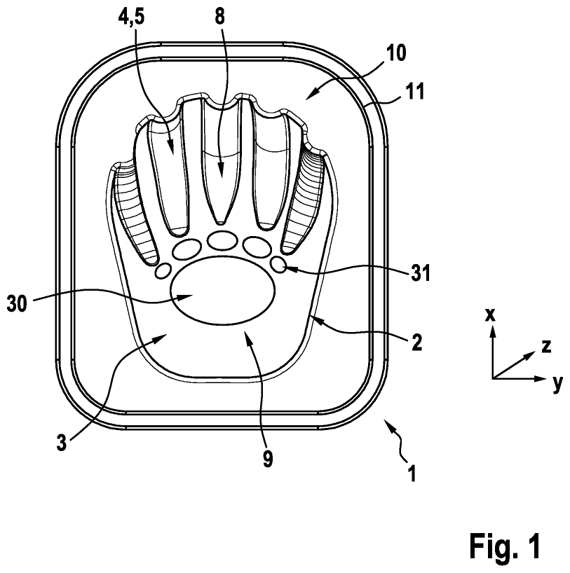

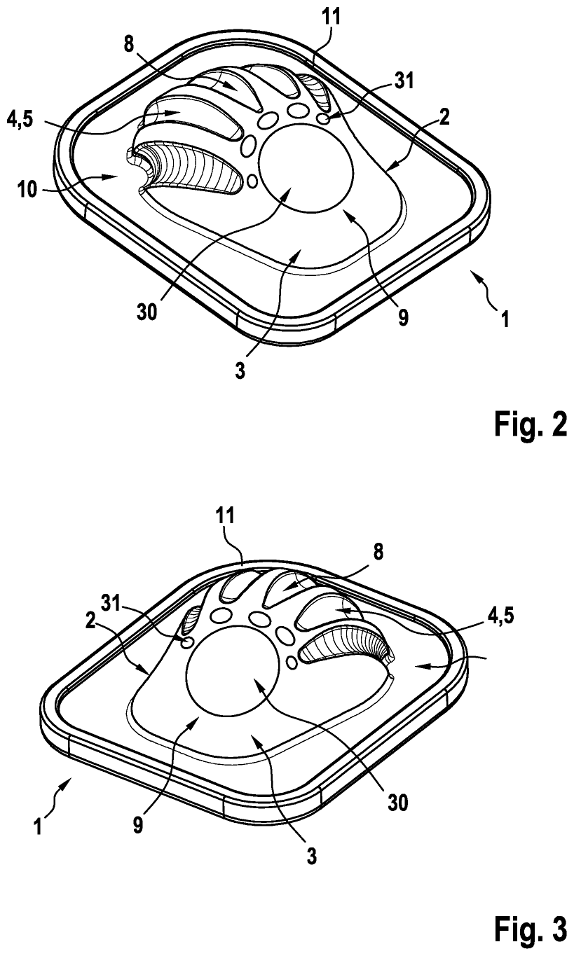

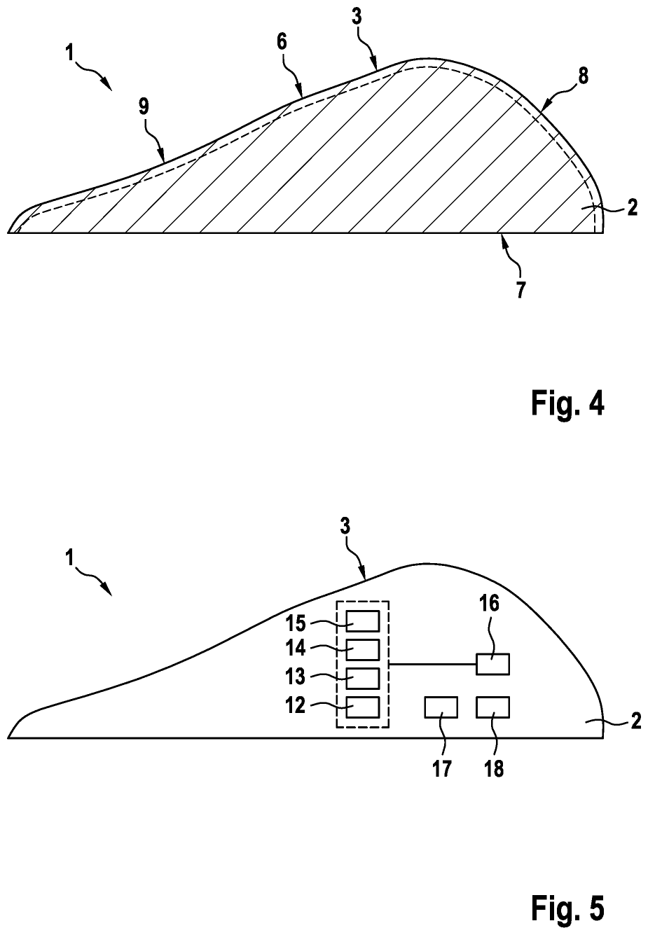

FIGS. 1 to 4 are a schematic perspective view of a support device 1 for supporting a body part (not shown) of a user according to an embodiment in a top view (FIG. 1), in a first perspective view from a first point of view (FIG. 2), in a second perspective view from a second point of view (FIG. 3) and in a sectional side view (FIG. 4).

In general, the support device 1 serves to support or mount at least one body part, i.e. a hand of a user shown in the embodiments in the figures.

The support device 1 comprises a support body 2. The support body 2, which can also be referred to as or considered to be a mounting body, also comprises a support region 3, which can also be referred to as or considered to be a mounting region, for supporting a body part of a user. The support region 3 is therefore understood to be the region of the support device 1 on which a user (directly) supports a body part when the support device 1 is used as intended. The support region 3 therefore constitutes the actual region of the support device 1 on which a user supports or mounts a body part when the support device 1 is used as intended. As shown in FIG. 1, the support region 3 typically forms at least one part of the (exposed) surface of the support device 1.

The support device 1 comprises a support body 2, which is designed to be configured in a predefined way, which comprises at least one surface. The surface of the support body 2 has three-dimensional contours or structures that are designed to be configured in a predefined way in respect of a particular body part of a particular user, which are formed in particular by the support region 3. The support region 3 can be formed in one piece with the support body 2 or formed as a separate part, which is or is to be connected to the support body 2 so as to form the support device 1.

The dimensions of the support body 2 and the support region 3 respectively are typically adapted to the dimensions of the body part to be supported in each case. When the support device 1 is used as intended, the support region 3 is therefore at least in portions, optionally completely, covered by the body part being supported in each case.

At least the support region 3 comprises a geometric-structural design or shape, designed to be configured in a predefined way, based on a piece of body part dimension information, from a plurality of pieces of predefined body part information selected from data describing support device dimension information, which describes the dimensions of a body part that is to be supported on the support device 1, wherein each support device dimension information describes predefined dimensions, assigned to at least one dimension of a body part, of a support device 1 which can be produced based on the respective support device dimension information. The support region 3 therefore comprises a geometric-structural design or shape, designed to be configured in a predefined way, which is formed or generated based on data described by a piece of support device dimension information.

The piece of support device dimension information, on which the data used for forming or generating of the support region are based on, is selected from a plurality of pieces of predefined support device dimension information, wherein each piece of support device dimension information, being assigned to at least a dimension such as length, width, height, etc., or at least a dimension ratio, such as a length-width-ratio, a length-height-ratio, a width-height-ratio, a length-width-height-ratio, etc. of a body part, describes predefined dimensions of the support device 1, which is or is to be produced based on the respective piece of support device dimension information. The selection of the piece of support device dimension information out of the plurality of pieces of support device dimension information is made based on a piece of body part dimension information, which describes dimensions of a body part that is to be supported on the support device 1 at least in portions, optionally completely.

The geometric-structural shape or form of the support region 3, which is configured in a predefined way, creates significant advantages in terms of manufacturing, since the support region 3 is not a component portion designed to be individually configured or a component portion designed to be individually configured. For the support region 3 is configured in a predefined way, i.e. it has a specific spatial-physical design, which is configured in a predefined way, and therefore a specific spatial form, configured in a predefined way, which is not individually configured with regard to a body part that is to be supported on the support device 1, even though it comprises support portions 4 formed in an abstracted or standardised shape with regard to certain characteristics of a respective body part that is to be supported, i.e. the number and arrangement of fingers of a (human) hand, as shown in the embodiments in the figures.

The support device 1 therefore does not have a geometric-structural design, which is designed to be individually configured, and thus no customisation other than a potential adjustment or scaling of the dimensions. The support device 1 or the support region 3 is therefore not an individual part that is individually configured for a particular body part of a particular user. Rather, the support device 1 or the support region 3 can be understood as a mass part that is easily produced in terms of manufacturing, with regard to specific dimensions of a body part, but otherwise entirely independent from individual characteristics of the respective body part. Due to the geometric-structural design or shape that is configured in a predefined way, the support device 1 or the support region 3 typically has a standardised geometric-structural design or shape, which is or is being adjusted only by scaling with regard to the dimensions of a body part to be specifically supported, if at all.

The geometric-structural design of the support region 3, with this being understood to be three-dimensional contours or structures, which are configured in a predefined way, of the surface of the support region 3, clearly constitutes a (negative) reproduction, which is abstracted in a predefined way or configured in a standardised way, of a body part that is to be supported on the support region 3. The abstraction or standardisation results in an accurate enough fit of the support region 3, despite of which it is not a fit of the support device 1 that is individually configured in a user-specific or body part-specific manner, this fit making it possible for a respective body part to be supported on the support region 3 in a relaxing or relaxed position specific to this body part.

Supporting a body part in a relaxing or relaxed position specific to this body part can both have a relaxing effect on the respective body part as well as on further body parts adjacent to this body part, such that a user's whole body can be brought into a relaxing or relaxed state when using the support device 1. In addition to the mere relaxing effect or as a consequence thereof, where necessary, therapeutic effects can also be brought about, since supporting the body part in a relaxing or relaxed position specific to this body part can e.g. have a therapeutic effect on the blood flow and/or oxygen supply to the respective body part. Positive effects can be achieved in particular in rheumatic body parts or limbs and/or in e.g. spastic body parts or limbs due to a stroke. The increase in the internal tension (tone) of the muscles of the respective body part, associated with spasticity, can be reduced (significantly in some cases) by (regular) use of the support device.

The support device 1 can be used without any help from a third party; therefore, the user can independently carry out therapeutic measures, i.e. for regulating tone, pain therapy, etc., and prepare themselves for corresponding therapeutic measures in the sense of self-help by using the support device 1 as intended.

Using the example of a spastic hand, the support device 1, which, in this respect, can be referred to as or considered to be a support for the heel of the hand for providing therapy to persons with a spastic or cramped hand, makes it possible for the hand to lie on the support device 1 in a loosened state, through which a state of relaxation is achieved.

With reference to FIG. 1, it is clear that the support region 3 may be integral or formed in one piece with the support body 2. The support region 3 forms an (exposed) surface of the support body 2 here. An (exposed) surface of the support body 2 therefore has an individually configured geometric-structural design.

On the basis of FIG. 4, as shown by the dashed line merely as example, it is conceivable for the support region 3 to be designed as a part that is separate from the support body 2 and, when required, is or can be connected, optionally in a detachable manner (without being damaged or destroyed), to the support body 2 so as to form the support device 1. In this variant the support body 2 and/or the support region 3 may comprise suitable, optionally corresponding connection elements (not shown), which allow for a stable, although typically (without being damaged or destroyed) detachable connection of the support body 2 with the support region 3 and vice versa. The support body 2 can e.g. comprise at least one first connection element, and the support region 3 at least one second connection element, wherein the at least one first connection element and the at least one second connection element are configured to interact with each other so as to form the support device 1. The at least one first connection element and the at least one second connection element may e.g. be configured to interact with one another in a form-fitting and/or force-locked manner so as to form the support device 1. It is also conceivable for the at least one first connection element and the at least one second connection element to be configured to interact with one another magnetically so as to form the support device 1.

With reference to FIGS. 1-4, it is clear that the support region 3 comprises several support portions 4, which are designed to be configured in a predefined way, which are configured to receive a particular body part or a limb portion of a body part that is supported or to be supported on the support region 3, or a limb that is supported or is to be supported on the support region 3. The support device 1 intended to support a particular body part may therefore comprise a plurality of support portions 4 that are configured in a predefined way specific to this body part. The support portions 4 are designed as receiving portions 5, which are configured in a predefined way, as shown in the embodiments in the figures.

As shown in the embodiments in the figures, the support region 3 comprises a specific number and arrangement of support portions 4, which are designed to be configured in a predefined way, which corresponds to the number and arrangement of the fingers of a respective hand of a user to be supported on the support device 1. The support portions 4 can therefore be referred to as or considered to be finger support portions.

Specifically, the support region comprises, in a (substantially) parallel arrangement, five finger support portions, which are designed to be configured in a predefined way, as shown in the embodiments in the figures, which (substantially) correspond to the number and arrangement of the fingers of a respective hand of a user that is to be supported on the support device 1. Respective finger support portions are also configured in a predefined way or designed in a standardised way. Specifically, respective finger support portions shown in the embodiments in the figures are exemplarily designed as receiving portions or recess portions, which are designed to be configured in a predefined or standardised way.

The finger support portions are clearly arranged and/or oriented in a manner extending in a first spatial direction (cf. x-axis in FIG. 1), especially in a distal spatial direction relative to a hand that is supported on the support device 1.

With reference to FIGS. 1-3 it is further clear that the support device 1 may comprise or include one or more further support portions 31, which are arranged or oriented in a manner transversely extending in particular toward the finger support portions, for receiving the end region of a (proximal) metacarpal region of a hand of a user that is to be supported on the support device 1. These further support portions 31 are also designed in a standardised way or configured in a predefined way and can specifically e.g. be designed as receiving portions or recess portions, which are designed to be configured in a predefined or standardised way.

Respective finger support portions can transition into a corresponding further support portion, in particular directly, i.e. communicate with a corresponding support portion, in particular directly.

With reference to FIGS. 1-4 it is clear that the support body 2 or the support body 3 comprise or include a base portion 6, which comprises several curvedly designed curved portions 8, 9, in particular relative to a non-curved base plane 7, which are in particular modelled on a hand. The curved portions 8, 9 are also designed to be configured in a predefined or standardized way and can, in particular in the metacarpal region, be designed as e.g. in particular pad-like or pad-shaped protrusions designed to be configured in a predefined or standardized way.

With reference to FIGS. 1-3 it is further clear that the proximal base portion 9, when the support device 1 is used as intended, may comprise at least one pad-like or pad-shaped curved portion 30, which may be configured to support a metacarpal region of a hand of a user that is supported on the support device 1.

As shown in the embodiments in the figures, the finger support portions are formed at the curved portion 8, which is curvedly designed in a distal ring segment-like or ring segment-shaped way when the support device 1 is used as intended.

In general, the support body 2 or the support region 3 comprises a base form, which is designed to be configured in a predefined way based on a hand that is to be supported on the support device 1.

As shown in the embodiments in the figures, the support body 2 is (optionally) arranged or formed on a base plate or carrier plate 10. The base plate or carrier plate 10 may be provided with at least one adhesion-increasing structuring such as a special surface structuring, e.g. a roughening, a rubber coating, etc., in order to ensure a stable support or mounting of the support device 1 on a surface.

The base plate or carrier plate 10, which is, as indicated, generally optional, can form a member of a carrier device (not otherwise specified) for carrying the support device 1. Along with a support body 2 arranged or designed on it, the base plate or carrier plate 10 can therefore be optionally detachable from, connected with, or connectable (without damaging or destroying) with e.g. a frame-like or -shaped carrier element 11 by attaching or inserting thereof, if necessary, by forming a corresponding carrier device. The base plate or carrier plate 10 or the carrier element 11 can comprise suitable, optionally corresponding connection elements which ensure a stable though typically (without damaging or destroying) detachable connection of the base plate or carrier plate 10 with the carrier element 11 and vice versa.

FIG. 5 is a schematic view of a support device 1 for supporting a body part (not shown) of a user according to a further embodiment in a side view.

With reference to the embodiment shown in FIG. 5, it is clear that the support device 1 may comprise a detection device 12, in particular integrated in the support body 2, which is configured to detect at least one chemical and/or physical variable of the support region 3 and/or the support body 2 and/or a body part supported on the support region 3. A corresponding detection device 12 may e.g. be configured to detect chemical parameters, such as the composition of the surface of a supported body part or a substance on the surface of a supported body part, and/or thermal parameters, such as the temperature of the support body 2 and/or a supported body part, and/or mechanical parameters, such as forces exerted on the support body 2 and/or the support region 3, in particular by means of a supported body part, i.e. in particular weight forces, pressure, stress, etc., and/or hydrometric parameters, such as moisture of a supported body part, etc. A corresponding detection device 12 may comprise one or more detection elements (not shown) that are e.g. designed as or comprise sensors, which are arranged or formed on or in the support body 2 and/or on or in the support region 3. A corresponding detection device 12 typically has suitable hardware-implemented and/or software-implemented evaluation and/or processing measures, such as suitable evaluation and/or processing algorithms, which allow for data-related evaluation and/or processing of respective detection signals supplied by the detection elements.

With reference to FIG. 5, it is also clear that the support device 1 may comprise a detection device 13, in particular integrated in the support body 2, which is configured to detect a body part supported on the support region 3. A corresponding detection device 13 may e.g. be configured to detect when a body part supported or to be supported on the support region 3 approaches or contacts the support region 3. A corresponding detection device 13 may comprise one or more detection elements (not shown) that are e.g. designed as or comprise sensors, in particular proximity sensors or contact sensors, which are arranged or formed on or in the support body 2 and/or on or in the support region 3. A corresponding detection device 13 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements. A corresponding detection device 13 may, however, also be configured to be identical to the above-mentioned detection device 12 for detecting a chemical and/or physical variable, since conclusions can be drawn on a body part supported on the support region 3 on the basis of respective detected chemical and/or physical variables.

With reference to FIG. 5, it is also clear that the support device 1 may comprise a detection device 14, in particular integrated in the support body 2, which is configured to detect at least one physiological variable, in particular the blood pressure, oxygen saturation or pulse, of a user supporting a body part on the support region 3. A corresponding detection device 14 may comprise one or more detection elements (not shown) that are e.g. designed as or comprise sensors, in particular blood-pressure, oxygen-saturation or pulse sensors, which are arranged or formed on or in the support body 2 and/or on or in the support region 3. Corresponding physiological variables may e.g. be detected via the contact region between the support region 3 and the respective body part supported on the support region 3. A corresponding detection device 14 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

With reference to FIG. 5, it is also clear that the support device 1 may comprise a detection device 15, which is configured to detect a movement of the support body 2 in at least one degree of freedom, in particular relative to a surface, and/or to detect a movement of a body part supported on the support region 3 in at least one degree of freedom, in particular relative to the support body 2. A corresponding detection device 15 may comprise one or more detection elements (not shown) that are e.g. designed as or comprise sensors, in particular motion sensors, which are arranged or formed on or in the support body 2 and/or on or in the support region 3. A corresponding detection device 15 typically has suitable hardware-implemented and/or software-implemented evaluation and/or processing measures, such as suitable evaluation and/or processing algorithms, which allow for data-related evaluation and/or processing of respective detection signals supplied by the detection elements.

As shown by the box formed by dashed lines, the detection devices 12-15 may also be combined in an optionally superordinate common detection device in terms of hardware and/or software.

With reference to FIG. 5, it is also clear that the support device 1 may comprise a transmission device 16, which is or can be assigned to the detection device(s) 12-15, in particular is integrated in the support body 2 and/or support region 3, and is configured to transmit respective detection information to at least one communication partner. A corresponding transmission device 16 may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

With reference to FIG. 5, it is also clear that the support device 1 may comprise a data storage device 17, in particular integrated in the support body 2 and/or the support region 3, which is configured to store at least one piece of detection information detected by means of the detection device 12-15. A data-transmission interface (not shown is typically assigned to a corresponding data storage device 17, by means of which data can be read from the data storage device 17 in a wired or wireless manner and/or by means of which data can be input into the data storage device 17.

With reference to FIG. 5, it is also clear that the support device 1 may comprise a signal-output device 18, in particular integrated in the support body 2, which is configured to generate at least one in particular acoustic and/or optical and/or haptic output signal, in particular on the basis of at least one piece of detection information generated by a detection device 12-15. Certain output signals can therefore be output via a corresponding signal-output device 18 by means of the support device 1. For example, an output signal indicating that a body part is properly supported on the support region 3 can be output. A corresponding output signal may be generated or have been generated for example on the basis of detection information supplied by at least one detection device 12-15, i.e. a piece of detection information describing that a body part has been detected as contacting the support region 3, for example. A corresponding signal-output device 18 typically comprises at least one signal-output element (not shown), which, depending on the specific signal to be output, may e.g. be designed as or comprise at least one acoustic signal-output element, i.e. a speaker, for example, as an optical signal-output element, i.e. a display element, light-emitting diode element, etc., for example, or as a haptic signal-output element, i.e. a vibration element, for example.

An output signal which can be output by means of a signal-output device 18, by way of example, may contain at least one piece of information relating to a user-side action and/or an evaluation of a user-side action, in particular on a user, of the support device 1. In this way, it is possible to assist a user in properly using the support device 1 by means of corresponding output signals containing corresponding information. For example, it can be output to a user by corresponding output signals that the support device 1 is ready to support a body part, and optionally also which body part, that a body part is properly supported or is not (yet) properly supported, or when/that a particular support duration, e.g. expedient for a therapeutic effect, has been reached or has not (yet) been reached, etc. Equally, output signals that encourage or motivate a user are conceivable, i.e. output signals that encourage or motivate a user, e.g. acoustically, optically or haptically, to use the support device 1, to assume and/or maintain a particular support position, etc. Similarly, output signals that assess, i.e. in particular praise or criticise a user, are conceivable, which assess a user or a particular user action, e.g. acoustically, optically or haptically, before and/or during and/or after use of the support device 1.

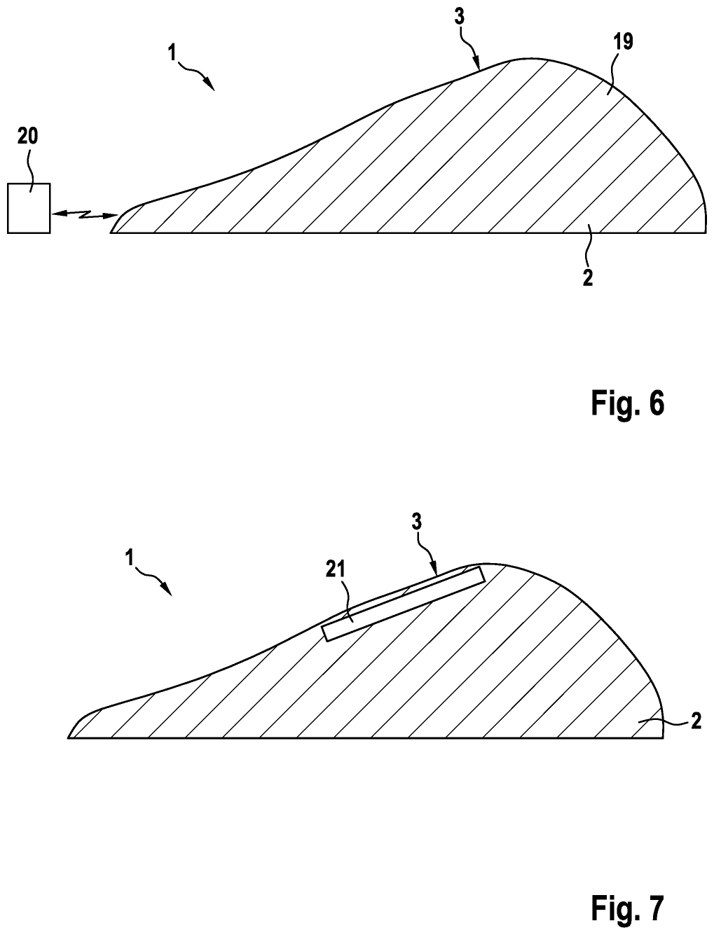

FIG. 6 is a schematic side view of a support device 1 for supporting a body part (not shown) of a user according to an embodiment in a side view.

With reference to FIG. 6, it is clear that the support body 2 may be integrated in an input apparatus 19 of a device 20, i.e. a terminal, for example, such as a computer, a multimedia apparatus, etc., that can be controlled by an input apparatus, or is designed as an input apparatus 19 of this kind. In this way, increased functional integration can be achieved, which also gives the user the option of carrying out particular actions, i.e. in particular inputs, controls, etc., using the body part while the body part is being supported. A specific example of a corresponding input apparatus 19 is, as shown in FIG. 6, an input element, e.g. designed as an (electronic) computer mouse, for making inputs, indicated by the arrow, into a computer, an input element, e.g. designed as a remote control, for making inputs into a multimedia apparatus, such as a television, an audio system, etc.

FIG. 7 is a schematic view of a support device 1 for supporting a body part (not shown) of a user according to an embodiment in a side view.

With reference to FIG. 7, it is clear that the support device 1 may comprise or include a temperature-control device 21, in particular integrated in the support body 2, for controlling the temperature of the support region 3 and/or the support body 2 at least in portions, optionally completely. The static or dynamic temperature control (understood in principle as both heating and cooling) of the support region 3 and/or the support body 2, made possible by means of a corresponding temperature-control device 21, can assist the desired relaxation of a respective body part that is being supported or can assist a therapeutic effect that can be achieved by a respective body part being supported.

A corresponding temperature-control device 21 may comprise a temperature-control duct structure (not shown) comprising a temperature-control duct through which a temperature-control medium, i.e. a gas or liquid, for example, can flow. A corresponding temperature-control duct structure may extend through the support body 2 or the support region 3, in particular in a planar manner, at least in portions, optionally completely. In particular, a corresponding temperature-control duct structure may extend, in particular with close contours, in the region of or below the (exposed) surface of the support body 2 or the support region 3 forming an actual support surface of the support body 2 or the support region 3.

Alternatively or additionally, a corresponding temperature-control device 21 may comprise at least one electrically operated temperature-control element (not shown). A corresponding electrically operated temperature-control element may e.g. be designed as or comprise at least one heating wire or a one-dimensional or multi-dimensional heating-wire structure. A corresponding temperature-control element may extend through the support body 2 or the support region 3, in particular in a planar manner, at least in portions, optionally completely. In particular, a corresponding temperature-control element may extend, in particular with close contours, in the region of or below the (exposed) surface of the support body 2 or the support region 3 forming an actual support surface of the support body 2 or the support region 3.

Alternatively or additionally, a corresponding temperature-control device 21 may comprise at least one temperature-control element, which performs an optionally reversible exothermic chemical reaction after activation. A corresponding temperature-control element may be arranged or formed in the region of or below the (exposed) surface of the support body 2 or the support region 3 forming an actual support surface of the support body 2 or the support region 3. A corresponding temperature-control element may comprise an activation element (not shown), i.e. a tab, a button, etc., by means of which it is possible to activate the temperature-control element, i.e. to initiate the exothermic reaction.

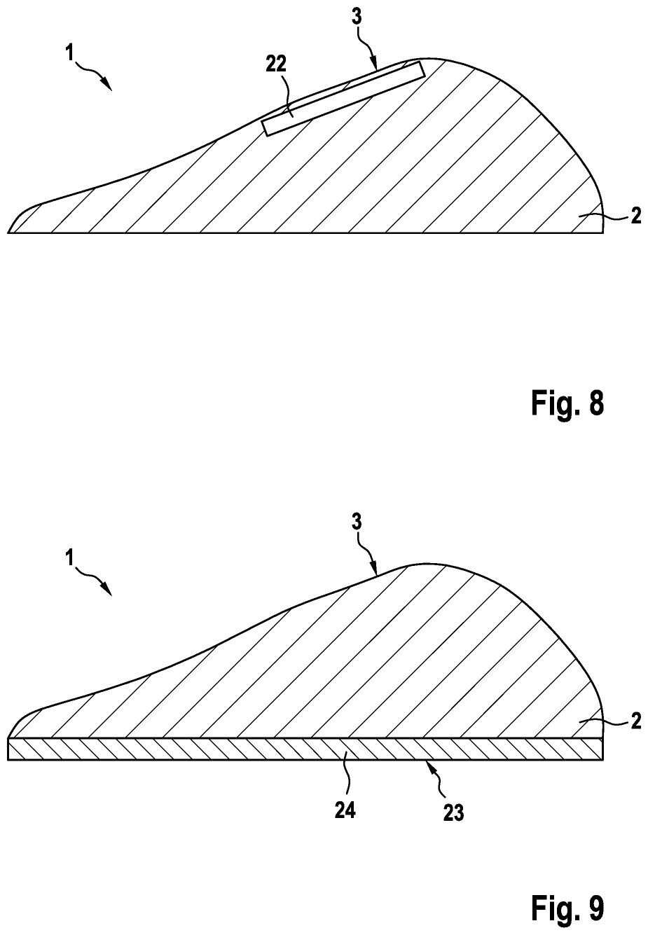

FIG. 8 is a schematic view of a support device 1 for supporting a body part (not shown) of a user according to further embodiments in a side view.

With reference to FIG. 8 it is clear that the support device 1 may comprise or include a venting device 22, which is in particular integrated in the support body 2 and/or support region 3, for ventilating at least in portions, optionally completely, a body part that is or is to be supported on the support device 3. The static or dynamic ventilation of the support body 2 or the support region 3, which is made possible my means of a corresponding venting device 22, may support the desired relaxation of a supported body part, respectively a therapeutic effect achieved by supporting the body part. The support properties can furthermore be improved in particular in supporting a body part over a longer period.

A corresponding venting device 22 may comprise a ventilation duct structure, which may comprise at least one ventilation duct, through which flows a ventilation medium, i.e. e.g. a gas. A corresponding ventilation duct structure may extend through the support body 2 or the support region 3, in particular in a planar manner, at least in portions, optionally completely. In particular, a corresponding ventilation duct structure may extend, in particular with close contours, in the region of or below the (exposed) surface of the support body 2 or the support region 3 forming an actual support surface of the support body 2 or the support region 3.

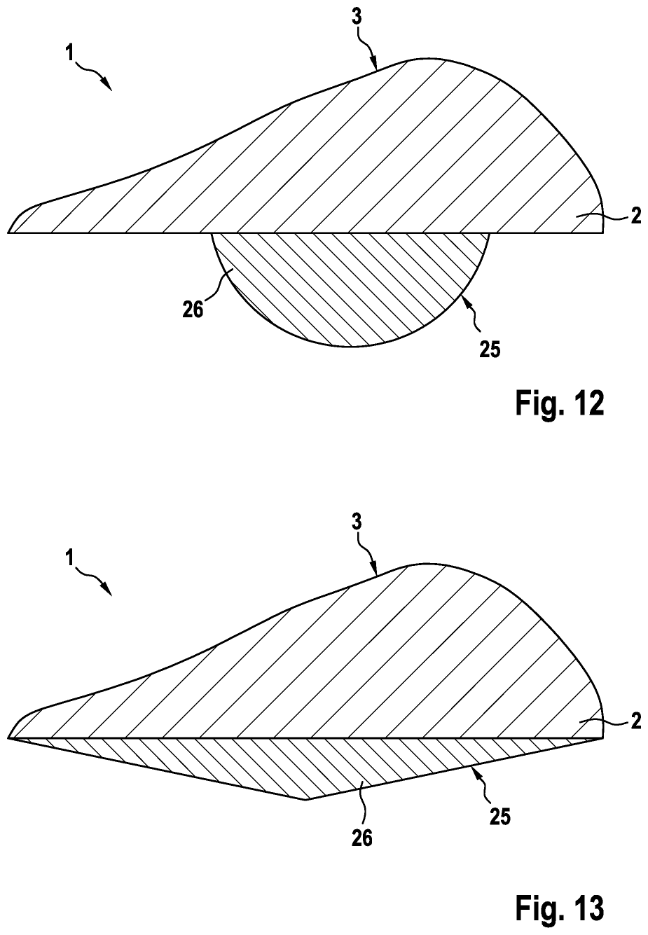

FIGS. 9-13 are schematic views of a support device 1 for supporting a body part (not shown) of a user according to further embodiments in a side view.

With reference to FIG. 9. it is clear that the support device 1 may comprise or include an attaching device 23 for temporarily attaching the support device 1 to or on a third object, in particular to or on a third object to be worn on a body of a user of the support device 1. The support device 1 may also be attached to third objects in such a way, i.e. in particular to mobile or portable third objects. Attachment of the support device 1 to corresponding third objects can in particular be such that the support device 1 may be in an orientation and/or position in which a desired orientation of a body part supported on the support region 3 is possible and can be maintained. A specific example for a third object is a third object which is to be worn on a body of a user, such as a piece of clothing. The support device 1 can therefore be attachable to or in a third object, such as a piece of clothing. It is, for example, conceivable, that the support device 1 be attachable in a pocket of a piece of clothing, such as a piece of clothing, which is to be worn on the upper body of a wearer, such as a jacket, a sweater, etc., or a piece of clothing to be worn on the lower body of a wearer, such as pants, a skirt, etc. The attaching device 23 may comprise one or more merely schematically implied attaching elements 24 which are configured for attachment (without being damaged or destroyed) of the support device 1 to a corresponding third object. Corresponding attaching elements 24 may e.g. allow attaching the support device 1 to a corresponding third object in a form-fitting and/or force-locked manner. A specific example for corresponding attachment elements 24 are therefore hook and/or loop elements (Velcro fastener elements).

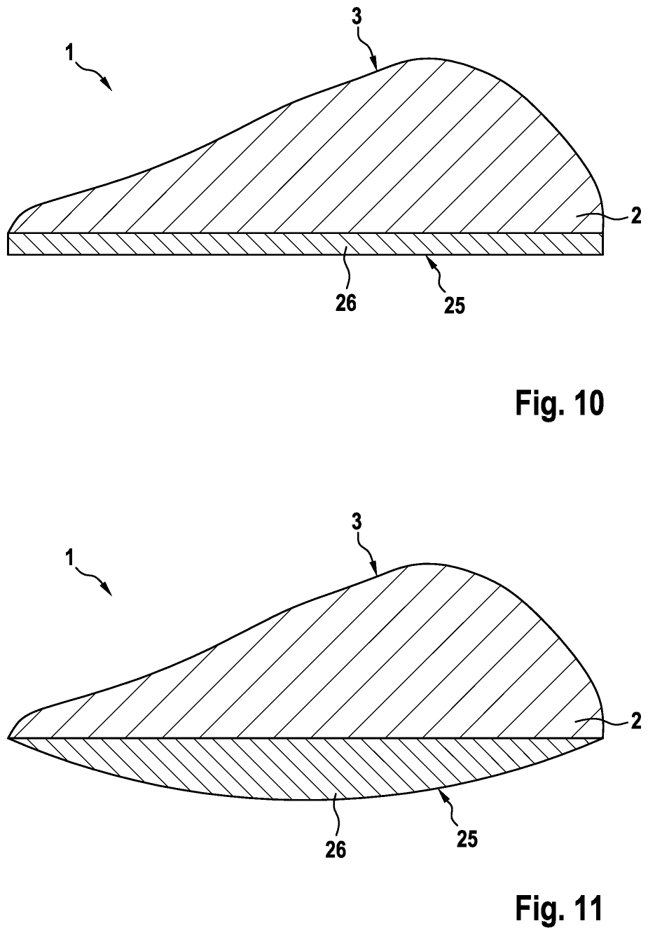

With reference to FIG. 10, it is clear that the support device 1 may comprise a support body 2 with a mounting portion 25 for mounting the support body 2 on a surface or a base. Depending on the design, the support body 2 can be mounted in a stable manner on a surface or a base, such as a table surface, via a corresponding mounting portion 25.

A corresponding mounting portion 25 may be provided with or comprise at least one material or corresponding material structure that provides non-slip support of the support body 2 on a surface or a base. For example, a corresponding material or a corresponding material structure may therefore be expedient for allowing the support body 2 to be supported in a stable manner on a surface or a base. A corresponding material or a corresponding material structure may e.g. be made of an elastomer material or an elastomer material structure or may comprise at least one elastomer material. In principle, both natural and synthetic elastomer materials or material structures are possible. In terms of manufacturing, thermoplastic elastomers may advantageously be used, for example.

A corresponding mounting portion 25 may be provided with or comprise at least one reversibly deformable material, in particular a reversibly deformable, flexible material, or a reversibly deformable material structure, in particular a reversibly deformable, flexible material structure. For example, a corresponding material or a corresponding material structure may therefore be expedient to allow any required modifications to the orientation of the support body 2 on a surface or a base, in particular relative to a neutral or reference orientation, which, for example, can make it easier or make it possible for users with spastic body parts or limbs to use the support device 1; this is in particular applicable if the spasticity in question or another anatomical malalignment requires a particular orientation of the support body 2, in particular relative to a neutral or reference orientation, in order to support the body part on the support region 3, in particular without causing any pain. A corresponding material or a corresponding material structure may e.g. be made of an elastomer or cellular material or an elastomer or cellular material structure or may comprise at least one elastomer material. In principle, both natural and synthetic elastomer or cellular materials or material structures are possible. In terms of manufacturing, optionally foamed, thermoplastic elastomers may advantageously be used, for example.

A corresponding mounting portion 25 may, as exemplarily shown in FIGS. 10-13, comprise a mounting-portion element 26 which in particular lies in a non-planar manner on a planar surface or base and makes it possible to mount the support body 2 that is mounted on the surface relative to the surface or base so as to be movable, in particular pivotally movable, in at least one degree of freedom. In this way, the support body 2 can be moved relative to a surface or a base, which may be advantageous for producing the most relaxed possible position of a respective body part and superordinate body parts. For example, an orientation of the support body 2 that can be modified compared with a reference orientation allows for (yet) more relaxing or relaxed support of a body part. A corresponding mounting-portion element 26 may, as shown in FIGS. 10-13, e.g. have a monogonal, polygonal, circular, round or pointed geometry. Likewise, movably mounting the support body 2 can provide training options, e.g. for building muscle, since by moving the support body 2 relative to the surface or the base, certain movement sequences associated with a training effect can be performed.

A corresponding mounting-portion element 26 may also be designed as a separate part and, when required, is or can be connected, optionally in a detachable manner (without being damaged or destroyed), to the support body 2 so as to form the support device 1. A corresponding, e.g. spherical, hemispherical, cylindrical or semi-cylindrical or pyramidal mounting-portion element 26 may e.g. be connected to the support body 2 by a Velcro fastener system or an interchangeable fastener system.

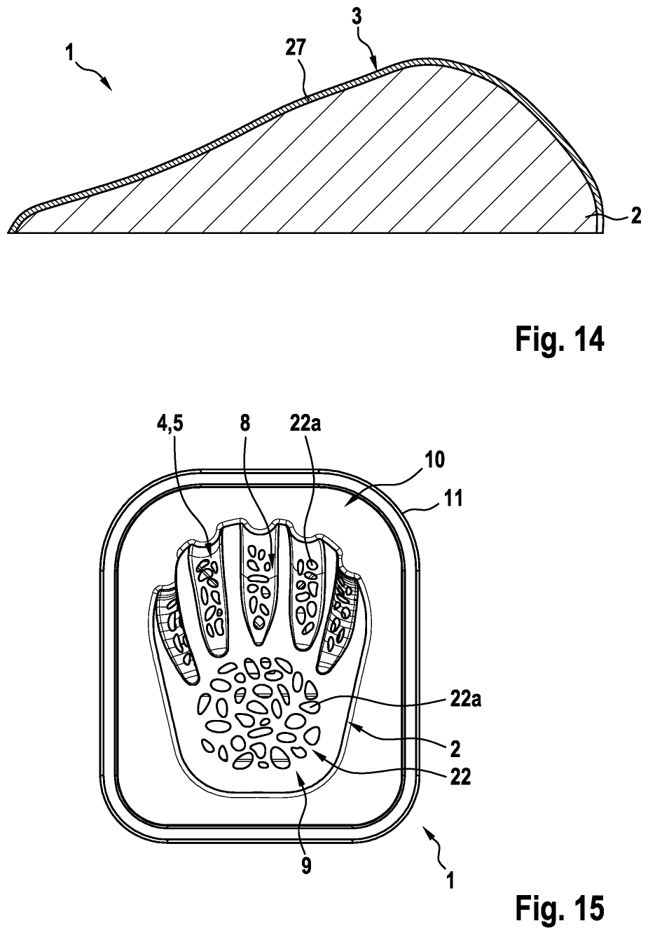

FIG. 14 is a schematic view of a support device 1 for supporting a body part (not shown) of a user according to a further embodiment in a side view.

With reference to FIG. 14, it is clear that the support device 1 may comprise a support element 27, which is or can be supported on the support region 3, in particular detachably (without being damaged or destroyed), so as to cover said support region at least in portions, optionally completely. A corresponding support element 27 may in particular be expedient from a hygiene viewpoint, since the support region 3 or the support body 2 is protected against direct contact with the respective body part and thus e.g. against deposits of skin, sweat, etc. A corresponding support element 27 may be made of a resiliently flexible material or a resiliently flexible material structure, such that the support element 27 does not impair the fit of the support region 3. A corresponding support element 27 may be made of an optionally washable, natural and/or synthetic fabric material or a natural and/or synthetic fabric material structure. Reference to leather or plastics materials is made merely by way of example,

FIG. 15 is a schematic view of a support device 1 for supporting a body part (not shown) of a user according to a further embodiment in a plan view.

The embodiment shown in FIG. 15 is also provided with a venting device 22 for ventilating the support region 3 and/or the support body 2 and/or a body part that is or is to be supported on the support device 1.

With reference to FIG. 15, it is clear that a corresponding ventilation duct structure of a ventilating device 22 may be formed by one or more bionic geometries, which are configured in a predefined way. In FIG. 15, the ventilation duct structure is implied by corresponding openings 22a of respective ventilation ducts. Corresponding openings 22a can be formed by e.g. by bore-like or bore-shaped cavities. Corresponding ventilation ducts can be clearly formed in the region of the base portion 6 as well as in the region of at least one support portion 4. However, it is of course conceivable that corresponding ventilation ducts are only formed in the region of the base portion 6 or in the region of at least one support portion 4.

Individual, several or all ventilation ducts can generally communicate with each other from their arrangement. Corresponding bionic geometries may support or improve the ventilation of a body part which is or is to be supported on the support device 1. It is therefore generally possible to form bionic geometries, which are configured in a predefined way, in the support body 2 in order to form a corresponding ventilation duct structure.

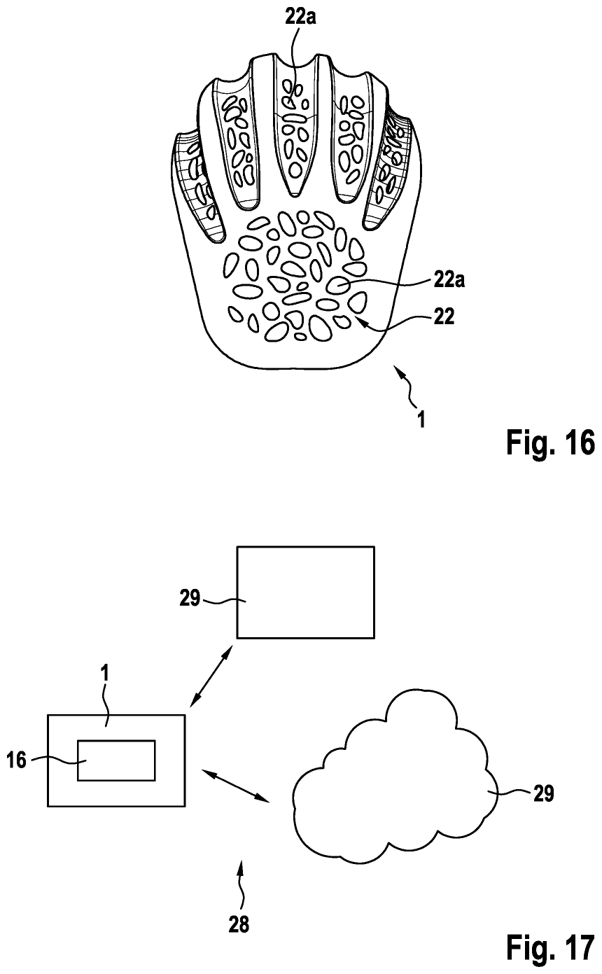

FIG. 16 is a schematic view of a support device 1 for supporting a body part (not shown) of a user according to a further embodiment in a plan view.

The embodiment show in FIG. 16 differs from the embodiment shown in FIG. 15 in that the support device 1 comprises no base plate or carrier plate 10 and therefore no carrier device for carrying the support device 1. As implied, this generally applies to all embodiments of the support device 1; all embodiments of the support device 1 can consequently be implemented without a corresponding carrier device for carrying the support device 1.

Although not shown in the drawings, the support device 1 may comprise a fixing device, which is or can be assigned to the support body 2 and/or the support region 3, for temporarily fixing a body part supported on the support region 3, in particular in an intended support position of the body part on the support region 3. Corresponding fixing may facilitate and maintain a desired orientation of a body part supported on the support region 3. In this way, a relaxation effect or therapeutic effect that can be produced by the support device 1 can be assisted. A corresponding fixing device can make it possible to fix a respective body part directly to the support body 2 or the support region 3; this may e.g. be implemented via a fixing element that is or can be attached to the support body 2 or the support region 3, such as a strap, a loop, etc., which surrounds the respective body part at least in portions, optionally completely, after being put on, such that the body part is fixed to the support body 2 or the support region 3. A tube-like or tubular fixing device is also conceivable, i.e. a fixing tube made of a fabric, for example, which can be pulled over the support device and the body part that is supported or is to be supported thereon and, in this way, a desired orientation of a body part supported on the support region is facilitated and maintained. A fixing device is also conceivable which makes it possible to fix a superordinate body part, i.e. an arm, for example, in a particular orientation when the body part to be fixed is a hand.

It is applicable to all the embodiments that the support region 3 and/or the support body 2 may be made of at least one foam material, i.e. in particular of an expanded particle foam material, such as an expanded plastic material. Corresponding foam materials reduce the weight of the support body 2 or the support region 3. Corresponding foam materials may additionally cause pleasant haptic properties of the support body 2 or support region 3. Further advantages of corresponding foam materials are the well manageable, cost-efficient machining of such.

It is further applicable to all the embodiments that the support region 3 and/or the support body 2 is made of or comprises at least one magnetisable or magnetic material. Magnetisable or magnetic materials, such as corresponding ferromagnetic metals, may have a positive effect on the blood flow through the corresponding body part, and this can promote therapeutic effects. Magnetisable or magnetic materials may be provided as a solid material or in the form of particles.

It is further applicable to all the embodiments that the support region 3 and/or the support body 2 is, alternatively or additionally, made of or comprises at least one antibacterial material. Antibacterial materials, such as antibacterial metals, may have a positive effect on the hygienic properties of the support region 3 and/or the support body 2. Antibacterial materials may be provided as a solid material or in the form of particles.

With regard to the production of the support device 1, i.e. the support body 2 and/or the support region 3, in principle any manufacturing methods, in particular moulding and/or shaping manufacturing methods, are taken into consideration.

In particular, both additive (build-up) manufacturing methods and subtractive (material-removing) manufacturing methods, i.e. in particular machining manufacturing methods, are taken into consideration.

FIG. 17 is a schematic view of a communication system 28 according to an embodiment. The communication system 28 comprises at least one support device 1, which comprises a transmission device 16, which is configured to transmit a piece of information to at least one communication partner 29 and/or to receive a piece of information transmitted by at least one communication partner 29, and at least one communication partner 29, which is configured to receive a piece of information transmitted by the at least one support device 1 and/or to transmit a piece of information to the at least one support device 1.

The communication partner 29 may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

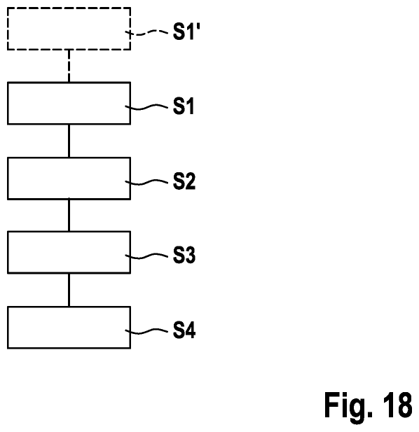

FIG. 18 is a schematic view of a method for producing a support device 1 for receiving a body part of a user according to an embodiment. The method comprises the following steps:

Step S1, in which a providing of a piece of body part dimension information, which describes the dimensions of a body part to be supported on a support device to be produced at least in portions, optionally completely, takes place.

Step S2, in which a providing of several pieces of support device dimension information, e.g. in a data storage device, takes place, wherein every piece of support device dimension information describes at least one predefined dimension, which is assigned to at least one dimension of a body part, of a support device that is or is to be produced based on the respective piece of support device dimension information.

Step 3, in which a selecting of a piece of support device dimension information based on the provided piece of body part dimension information takes place.

Step 4, in which a producing of a support device 1 based data containing or describing the selected piece of support device dimension information takes place.

Corresponding data, on the basis of which the support device 1 is produced according to the method, can therefore include production data of a production plant usable for or be used for producing the respective support device 1 or be converted into such production data.

The selection can be done according to the method by matching or comparing the provided piece of body part dimension information with respective body part dimension information assigned to respective support device dimension information, wherein a piece of support device dimension information can be chosen, whose assigned piece of body part dimension information is equal or similar, in particular within the scope of a tolerance range which can be or is predefined, to the provided piece of body part dimension information.

A piece of body part dimension information, which is or is to be provided according to the method, may be done by detecting, in particular optically, a body part of a user that is to be supported on the support device 1. According to the method, an in particular optical detection of a body part of a user that is to be supported on the support device, as well as a generation of a piece of body part dimension information, which is to be provided, can therefore be made based on a piece of detection information generated by the detection (Step S1′). As implied, the detection can be made in particular optically, so that a detection can be made e.g. by means of a detection device in the form of a camera device or a video device. A corresponding camera device or video device constitute a member of a user- or producer-side, in particular mobile, end-device, such as a mobile computer (laptop), a smartphone, a tablet, smart glasses, etc.

Individual features, multiple features or all the features disclosed in connection with a particular embodiment may be combined with individual features, multiple features or all the features disclosed in connection with at least one other embodiment.

A key aspect relates to a support device for supporting at least one body part of a user at least in portions, optionally completely, comprising a support body, which comprises at least one support region for supporting at least one body part of a user at least in portions, optionally completely, wherein at least the support region comprises at least in portions, optionally completely a geometric-structural design or shape, designed to be configured in a predefined way, based on a piece of body part dimension information, from a plurality of pieces of predefined body part information selected and formed or generated from data describing support device dimension information, which describes the dimensions of a body part that is to be supported on the support device 1 at least in portions, optionally completely, wherein each support device dimension information describes predefined dimensions, assigned to at least one dimension of a body part, of a support device 1 which can be produced based on the respective support device dimension information.

1. A support device for supporting at least one body part of a user at least in portions comprising a support body which comprises at least one support region for supporting at least one body part of a user at least in portions, wherein at least the support region has, at least in portions, a geometric-structural design or shape which is designed to be configured in a predefined way which is designed or generated on the basis of data described by a piece of support device dimension information, wherein:
the piece of support device dimension information being the basis of the data used for designing or generating the support device is selected from a plurality of pieces of predefined support device dimension information; and
the selecting of support device dimension information out of the plurality of support device dimension information takes place on the basis of a piece of body part dimension information, which describes the dimensions of a body part that is to be supported on the support device at least in portions.

2. The support device according to claim 1, wherein the support body or the support region comprises one or more support-region portions configured in a predefined way, which are in particular designed as receiving portions and are configured to receive a particular body part of a body part that is supported or is to be supported on the support region.

3. The support device according to claim 2, wherein the support body or the support region comprise a specific number and/or a specific arrangement of receiving portions designed to be configured in a predefined way, which corresponds to the number and/or the arrangement of the limb portions of a respective body part of a user that is to be supported on the support device.

4. The support device according to claim 3, wherein the support body or the support region comprises at least two finger support portions designed to be configured in a predefined way, in parallel arrangement, which corresponds to the number and arrangement of the fingers of a hand of a user that is to be supported on the support device.

5. The support device according to claim 3, wherein the finger support portions are arranged and/or oriented extending in a first spatial direction relative to a hand supported on the support device.

6. The support device according to claim 5, comprising at least one further support portion, arranged or oriented extending transversely to the finger support portions, for supporting an end region of a metacarpal region of a hand of a user that is to be supported on the support device.

7. The support device according to claim 5, wherein the finger support portions transition into the further support portion.

8. The support device according to claim 1, wherein the support body or the support region comprises a base portion which comprises one or several curvedly formed curved portions relative to a non-curved base plane, which are modelled on a hand.

9. The support device according to claim 4, wherein the finger support portions and/or at least one further support portion are formed in at least one ring segment-like or ring segment-shaped curvedly formed curved portion.

10. The support device according to claim 3, wherein the base portion comprises a pad-like or pad-shaped curved portion which is configured to support a metacarpal region of a hand of a user that is or is to be supported on the support device when the support device is in a state where it is used as intended.

11. The support device according to claim 2, wherein the support body or the support region comprises a base form, which is designed to be configured in a predefined way, modelled on a body part to be supported on the support device.

12. The support device according to claim 2, wherein the support body is arranged or formed on a base plate.

13. The support device according to claim 1, comprising a detection device integrated in the support region and/or the support body, which is configured to detect at least one chemical and/or physical variable of the support region and/or the support body and/or a body part supported on the support region.

14. The support device according to claim 13, comprising a transmission device, which is or can be assigned to the detection device, integrated in the support region and/or the support body, which is configured to transmit a piece of detection information describing a chemical and/or physical variable detected by means of the detection device to at least one communication partner.

15. The support device according to claim 1, comprising a detection device integrated in the support region and/or the support body, which is configured to detect a body part supported on a support region.

16. The support device according to claim 1, comprising a transmission device integrated in the support region and/or the support body, which is configured to detect at least one physiological variable, in particular the blood pressure, oxygen saturation or pulse, of a user supporting a body part on the support region.

17. The support device according to claim 1, comprising a detection device integrated in the support region and/or the support body, which is configured to detect a movement of the support body in at least one degree of freedom relative to a surface, and/or to detect a movement of a body part supported on the support region in at least one degree of freedom relative to the support body.

18. The support device according to claim 1, comprising a data storage device integrated in the support region and/or the support body, which is configured to store at least one piece of detection information detected by means of the detection device.

19. The support device according to claim 1, comprising a signal-output device integrated in the support region and/or the support body, which is configured to generate at least one acoustic and/or optical and/or haptic output signal on the basis of at least one piece of detection information generated by the or a detection device.

20. The support device according to claim 1, wherein the support body is integrated at least in portions, in an input apparatus of a device that can be controlled by an input apparatus, or is designed as an input apparatus.

21. The support device according to claim 1, comprising a temperature-control device integrated in the support region and/or the support body, for controlling the temperature of the support region and/or the support body and/or of a body part that is or is to be supported on the support device at least in portions.

22. The support device according to claim 1 comprising a venting device integrated in the support region and/or the support body, for ventilating the support region and/or the support body and/or a body part that is or is to be supported on the support device at least in portions.

23. The support device according to claim 1, comprising a fixing device for temporarily fixing a body part supported on the support region in an intended support position and/or support orientation of the body part on the support region.

24. The support device according to claim 1, comprising an attaching device for temporarily attaching the support device on or to a third object that is to be worn at or on a body of a user of the support device.

25. The support device according to claim 1, wherein the support body comprises at least one mounting portion for mounting the support body on a surface, wherein the mounting portion comprises at least one mounting-portion element which lies in a non-planar manner on a planar surface and makes it possible to mount the support body that is mounted on the surface relative to the surface so as to be pivotally movable, in at least one degree of freedom.

26. The support device according to claim 1, wherein the support region is designed as a part that is separate from the support body so as to form the support device and, when required, can be connected to the support body.

27. The support device according to claim 1, comprising at least one support element, which is or can be supported on the support region, so as to cover said support region at least in portions.

28. The support device according to claim 1, wherein the support body and/or the support region is made of or comprises a foam material, at least in portions; and/or
the support region and/or the support body is made of or comprises at least one magnetisable or magnetic material; and/or
the support region and/or the support body is made of or comprises at least one antibacterial material.

29. A communication system comprising:
at least one support device according to claim 1, which comprises a transmission device configured to transmit a piece of information to at least one communication partner and/or to receive a piece of information transmitted by at least one communication partner; and
at least one communication partner configured to receive a piece of information transmitted by the at least one support device and/or to transmit a piece of information to the at least one support device.

30. A method for producing a receiving device for supporting at least one body part of a user at least in portions, comprising the steps of:
providing a piece of body part dimension information describing the dimensions of a body part to be supported on a support device to be produced at least in portions;
providing several pieces of support device dimension information, wherein each piece of support device dimension information describes at least one dimension assigned to at least one dimension of a body part of a support device produced or to be produced on the basis of the respective piece of support device dimension information;
selecting a piece of support device dimension information based on a provided body part dimension information; and
producing a support device on the basis of the selected piece of support device dimension information.

31. The method according to claim 30, wherein the selecting comprises matching or comparing the provided body part dimension data with respective body part dimension information assigned to respective support device dimension information, wherein a piece of support device dimension information is selected, whose assigned piece of body part dimension information is equal to the provided body part dimension information or is similar to it within the scope of a tolerance range that is predefined or may be predefined.

32. The method according to claim 30, comprising detecting, a body part of a user on the support device and generating a piece of body part dimension information that is to be provided, on the basis of a piece of detection information generated by the detecting.
